(19) United States
(12) Patent Application Publication
(10) Pub. No.: US 2010/0215833 A1
(43) Pub. Date: Aug. 26, 2010

(54) COATING FOR MEDICAL DEVICE AND METHOD OF MANUFACTURE

(76) Inventor: Lothar Sellin, Aachen (DE)

Correspondence Address: Paul D. Greeley;Ohlandt, Greeley, Ruggiero & Perle, L.L.P., One Landmark Square, 10th Floor, Stamford, CT, 06901-2682, US

(21) Appl. No.: 12/536,379

(22) Filed: Aug. 5, 2009

Related U.S. Application Data
(63) Continuation-in-part of application No. PCT/IB2009/000531, filed on Mar. 16, 2009.

(30) Foreign Application Priority Data
Feb. 26, 2009 (DE) 10 2009 010 401.1

(51) Int. Cl. A61F 2/06; A61M 29/00; C07C 11/00; C09D 7/00; A61K 47/00; A61K 31/337; A61K 31/436

(52) U.S. Cl. 427/2.25; 604/96.01; 623/1.15; 427/2.3; 585/16; 106/285; 106/218; 514/772; 514/449; 514/291

(57) ABSTRACT

The invention relates to a medical device with a base body and a coating, wherein the coating exhibits film or lacquer-forming terpenoids. In addition, the coating of the medical device can exhibit at least one application-specific additive.

The invention further relates to a method for manufacturing such a device.

The invention relates to a medical device with a coated base body and a method for its manufacture.

Medical devices with various coatings are known from prior art. They are used in human and veterinary medicine, but in particular in cardiology as well.

Numerous coatings with terpenoids are also known from prior art.

Terpenoids have the property of adhering well to surfaces, as well as being abrasion resistant and readily miscible with other materials. Therefore, in addition to purely technical applications, terpenoids also have a wide range of applications as a coating material for orally applied medications, food supplements and cosmetics. In terms of cosmetics, numerous applications for shellac have been published in particular in the area of dermatology.

JP 300539737 describes a method for manufacturing a surface coating of powder, granules or tablets, which contain active ingredients, in particular selective proteins, in order to achieve initial release in the duodenum, not in the stomach. Other than the usual synthetic, the used materials also include zein and shellac.

A combination of shellac and carbonates for oral applications is disclosed in JP 10218795, generally for the area of (health) foods and medicine.

JP 6203620 also involves the use of shellac for coating hemoglobin-containing basic materials, so that they can be orally applied for treating iron deficiency in cases of anemia.

EP 167 90 78 describes the use of shellac in the treatment of diabetes and gangrene-based wounds, psoriasis and similar skin diseases.

Selected, naturally occurring resins like pine oil, chamfer and colophonium, the constituents of which belong to the class of terpenoids, are used in CN 126199 as raw materials for manufacturing multifunctional, fast-acting medical plasters. Descriptions center especially on the moisture-eliminating, analgesic, anti-inflammatory and detumescent effect.

Patent EP 1666019 discloses coatings of natural teeth with shellac and colophonium to improve appearance and fight caries. In addition, various pigments are added to the tooth lacquer for beautification purposes.

In WO 03034944, stents are coated with a hemocompatible layer, which have one or more additional layers, wherein these in turn contain at least one anti-proliferatory and/or anti-inflammatory and, if necessary, anti-thrombotic agent. The active agents here also include terpenoids, e.g., which are used in cancer treatment, among many others.

Patent Application DE 107 34 544 A1 describes the addition of shellac dispersions to O/W or W/O emulsions or hydrogels, in order to improve how the skin feels with respect to conventional emulsions and gels (preventing an oily or sticky sensation) while simultaneously increasing hygrostability and achieving stable emulsions. Shellac is used extensively as a coating material under the designation E 904 in the area of food additives.

The object of the present invention is to provide improved medical devices.

This object is achieved with a medical device having a base body and a coating, wherein the coating exhibits film or lacquer-forming terpenoids. The physical and antibacterial properties are here used, as are the typical release characteristics. The present coating with film or lacquer-forming terpenoids protects the medical device, but also offers an opportunity to become functionally effective. This functional aspect relates to material properties like strength and elasticity of the coating, and the actions originating from the coating, e.g., antibacterial effects.

The medical device is advantageously a catheter.

In particular, it can be a balloon catheter, especially a PTA or PTCA catheter. Balloons can include all balloons that are used in the field of medicine, and able in some manner to apply, i.e., dispense, medication in some manner to prevent or eliminate a restenosis.

In addition, a stent can be mounted on the balloon catheter, in particular a coronary or vascular stent. While it might be medically necessary to treat a coronary restenosis purely with a coated balloon catheter, many indications require that a stent be placed as well. The latter is then set in the coronary stenosis via the same coated balloon catheter as the stent carrier system. The active agent is here dispensed from the stent carrier balloon into the vascular wall. This enables stenting assisted by an active agent.

The medical device can also be a vascular prosthesis. The latter can exhibit the coating on the inside.

The medical device can also be a stent. The latter can exhibit the coating both on the inside and outside.

The terpenoid materials contained in the coating can be of natural, synthetic or semi-synthetic origin.

It has proven especially advantageous for the terpenoids to belong to one of the following substance classes, or contain mixtures thereof: monoterpenes, sesquiterpenes, diterpenes, seterterpenes, triterpenes and tetraterpenes.

In addition, the coating can contain additional materials of organic or inorganic origin.

It is especially advantageous for the inorganic materials to be oxides and/or salts. Possible salts include phosphates, sulfates, silicates, carbonates or mixtures thereof. Adding these inorganic materials plays a special role in the integration of the implants, and is intended to stimulate growth by providing corresponding ions.

With respect to organic materials, lipids or components thereof have proven themselves advantageous. These can be carbonic acids, their derivatives or substitution products. Adding these organic materials produces a positive change in the coating elasticity. The fabricated balloon coating becomes more compressively elastic, and hence less brittle.

It has been found that in particular the terpenoids shellol acid and abietic acid or their naturally occurring form as shellac and colophonium possess the desired physicochemical properties as a coating material for balloon catheters.

However, the resins dammar and mastic can likewise be used for coating purposes, wherein their constituents also represent terpenoids.

An application-specific additive can be advantageously incorporated into the coating material. The application-specific additives enable a broad pharmacological effect of the coating. The coating with film and lacquer-forming terpenoids here leads to an acceleration of active agent penetration, for example through the vascular wall, which yields improved bioavailability in the tissue. This results in an enhanced therapeutic effectiveness.

For example, the additive can be an anti-proliferatory, anti-inflammatory and/or anti-bacterial agent. One example for an anti-bacterial agent would be copper.

The additive can also be a medication for reducing or preventing restenosis.

The additive can be an anti-mitotic agent. The latter prevents cellular division, and hence cell division [translator's note: German repeats cell division], thereby developing an anti-restenotic effect.

In particular, the anti-mitotic agent can be paclitaxel.

An additive can also be an immunosuppressor. In particular if the medical device is a vascular prosthesis or stent, this can make sense, since this prevents a rejection by the body.

In particular immunosuppressors from the group of linus derivatives can be used, such as rapamycin (sirolimus), everolimus, zoratolimus, biolimus, neolimus, and deforolimus.

Finally, an additive can also be a growth factor inhibitor. This also inhibits cell division, thereby producing an anti-restenotic effect.

The additive can be incorporated in the form of a solid nano- or micro-particles or capsules. In addition to the direct incorporation of bioactive substances like antibiotics, cytostatics, hormones or growth factors or a combination of these substance classes, the bioactive substances can be immobilized before use. The immobilization/encapsulation method used can achieve a direct release of active substances via the balloon catheter.

Another aspect of the invention involves a method for manufacturing a medical device. The process for manufacturing the coating is advantageously implemented beginning with a preceding step, in which the terpenoid components are converted into a homogeneous solution. Preferred solvents here include lower alcohols, ketones and esters. In particular ethanol can be used. After the desired concentration has been set, the inorganic and/or organic materials and/or an application-specific additive can be incorporated.

Once all components have been combined, homogenization takes place using various agitating techniques (agitating mills, dispersers) depending on the viscosity of the compound. In a next step, the coating solution can be applied to the surface of the medical device via immersion, spraying, pouring, insertion or spreading, and dried in a subsequent step.

In addition, the coating material can be applied to the surface of the balloon catheter via pressure reduction, e.g., evaporation in a vacuum chamber.

In this case, the application of the method is not limited just to static surfaces. Rather, the coating material can be applied to the surface, while the medical device rotates.

Two exemplary embodiments will be described below, wherein the invention is not limited to the exemplary embodiments described here.

In a first exemplary embodiment, 5 g of shellac are dissolved in 100 ml of ethanol under mixing for 24 hours. 2% stearin (w/w) is added to this solution. The solution is blended for another 4 h at 30° C. The balloon catheter is immersed into this solution. Repeated immersion yields the desired layer thickness. This is followed by drying for 1 h at 40° C.

In a second exemplary embodiment, 5 g of shellac are dissolved in 100 ml of ethanol under mixing for 24 hours. The solution is applied to the balloon catheter using a spraying technique. A cytostatic is then applied to the suspension via immersion or spraying. The cytostatic is released after a few minutes (1-2 min).

1. A coating for medical device comprising film or lacquer-forming terpenoids.

2. (canceled)

3. (canceled)

4. (canceled)

5. (canceled)

6. (canceled)

7. The coating according to claim 1, wherein the terpenoids are of natural, synthetic or semi-synthetic origin.

8. The coating according to claim 1, wherein the terpenoids belong to one of the following substance classes or contain mixtures thereof: monoterpenes, sesquiterpenes, diterpenes, sesterterpenes, triterpenes and tetraterpenes.

9. The coating according to claim 1, comprising additional materials of organic or inorganic origin.

10. The coating according to claim 9, wherein the materials of inorganic origin are oxides and/or salts.

11. The coating according to claim 9, wherein the materials of inorganic origin are phosphates, sulfates, silicates, carbonates or mixtures thereof.

12. The coating according to one of claim 9, wherein the materials of organic origin are lipids or components thereof.

13. The coating according to claim 9, wherein the materials of organic origin are carbonic acids, its derivatives or substitution products.

14. The coating according to claim 1, wherein the coating exhibits shellol acid.

15. The coating according to claim 1, wherein the coating exhibits abietic acid.

16. The coating according to claim 1, wherein the coating exhibits shellac.

17. The coating according to claim 1, wherein the coating exhibits colophonium.

18. The coating according to claim 1, wherein the coating exhibits the resins dammar and/or mastic.

19. The coating according to claim 1, wherein the coating exhibits at least one application-specific additive.

20. The coating according to claim 19, wherein an additive is an anti-proliferatory, anti-inflammatory and/or anti-bacterial agent.

21. The coating according to claim 19, wherein an additive is a medication to reduce or prevent restenosis.

22. The coating according to one of claim 19, wherein an additive is an anti-mitotic agent.

23. The coating according to claim 22, wherein the anti-mitotic agent is paclitaxel.

24. The coating according to claim 19, wherein an additive is an immunosuppressor.

25. The coating according to claim 19, wherein an additive is an immunosuppressor from the group of linus derivatives, in particular rapamycin (sirolimus), everolimus, zoratolimus, biolimus, neolimus, and deforolimus.

26. The coating according to claim 19, wherein an additive is a growth factor inhibitor.

27. The coating according to claim 19, wherein the additive exhibits the shape of solid nano- or micro-particles or capsules.

28. A method for manufacturing a coating for a medical device comprising the steps of
adding solvents to terpenoid components
homogenizing the terpenoid components to form a homogeneous terpeniod solution; and
applying the homogeneous terpenoid solution to the surface of a medical device.

29. The method according to claim 28, wherein the coating material is separated out onto the surface of the device via pressure reduction.

30. The method according to claim 28, wherein the solution is applied to the surface as the medical device rotates.

31. The coating according to claim 1, wherein the device is one of a catheter, a vascular prostheses or a stent.

32. The coating according to claim 1, wherein a stent is mounted on the balloon catheter.

33. The coating according to claim 31, wherein the catheter is a balloon catheter.
